(19) United States
(12) Patent Application Publication
(10) Pub. No.: US 2026/0354687 A1
(43) Pub. Date: Nov. 12, 2026

(54) System and Method for Soft Tissue Assessment of a Joint

(71) Applicant: OMNIlife science, Inc., Raynham, MA (US)

(72) Inventor: Christopher Plaskos, Plymouth, MA (US)

(73) Assignee: OMNIlife science, Inc., Raynham, MA (US)

(21) Appl. No.: 19/673,883

(22) Filed: May 11, 2026

Related U.S. Application Data
(60) Provisional application No. 63/803,108, filed on May 9, 2025.

(51) Int. Cl. A61B 5/00; A61B 5/11

(52) U.S. Cl. CPC A61B 5/0057; A61B 5/11

(57) ABSTRACT

An orthopedic system for conditioning a soft tissue structure is provided. The system includes a distraction device, a sensor and a controller. The distraction device includes a first connector configured to engage a first anatomical structure associated with a soft tissue structure, a second connector configured to engage a second anatomical structure associated with the soft issue structure, and a displacement mechanism for applying a force to the connectors. The sensor is configured to generate parameter measurement data associated with the soft issue structure and/or the distraction device upon application of the displacement mechanism. The parameter measurement data comprises a force and/or a displacement of the soft tissue structure. The controller is configured to receive parameter measurement data from the sensor, determine a characteristic parameter of the soft tissue structure based on the parameter measurement data, and control the displacement mechanism in response to the determined characteristic parameter.

### CROSS-REFERENCE TO RELATED APPLICATION

This application claims the benefit of U.S. Provisional Application No. 63/803,108; filed May 9, 2025; entitled SYSTEM AND METHOD FOR SOFT TISSUE BALANCING OF A JOINT; which application is hereby incorporated by reference in its entirety for all purposes.

### FIELD OF DISCLOSURE

The present disclosure relates to orthopedic surgical systems and methods, and more particularly to a system and method for soft tissue assessment of a joint using a distraction device configured to control and measure various properties of the soft tissue of a joint pre-operatively or intra-operatively.

### BACKGROUND

Orthopedic surgical procedures frequently involve the manipulation, repair, or reconstruction of soft tissue structures of a joint such as ligaments, tendons, and joint capsules. These surgical procedures may include ligament reconstruction surgeries, such as anterior cruciate ligament (ACL) reconstruction in the knee, as well as joint arthroplasty procedures in the knee, shoulder, hip, ankle, and spine. The success of such surgical procedures often depends on achieving appropriate tension and balancing of the soft tissue surrounding the joint i.e., soft tissue structures.

For example, during ligament reconstruction procedures, a graft is typically used to replace a damaged or torn ligament. The graft may be secured to the bones of the joint through tunnels drilled in the bone, and the tension applied to the graft during fixation can affect the stability and function of the reconstructed joint. Similarly, in joint arthroplasty procedures, the balance of soft tissues around the joint e.g., the collateral ligaments, can influence the kinematics, stability, and longevity of the implanted prosthesis.

Soft tissue structures exhibit viscoelastic properties, meaning their mechanical behavior is dependent on both time and loading history. When subjected to loading (e.g., load forces), soft tissues may undergo creep, where the tissue deforms over time under constant stress, or stress relaxation, where the stress in the tissue decreases over time under constant strain. These time-dependent properties can affect the tension in a graft or soft tissue structure after surgical fixation.

Assessing joint laxity and soft tissue tension pre-operatively and intraoperatively is challenging. Surgeons may rely on manual techniques and subjective assessments to evaluate joint stability and soft tissue balance.

Various surgical approaches exist, for example, for joint arthroplasty procedures, including mechanical alignment, anatomical alignment, kinematic alignment, and gap balancing techniques. Each approach may involve different considerations for bone resection depth, position and orientation, and soft tissue management. The selection of implant size and positioning can also affect the balance of forces across the joint and the tension in surrounding soft tissue structures.

Thus, there is still a need for systems and methods that can assist in measuring and controlling tension of soft tissue structures of a joint.

### SUMMARY

This summary is provided to introduce a selection of concepts in a simplified form that are further described below in the detailed description. This summary is not intended to identify all key features or all essential features of the claimed subject matter, nor is it intended to be used as an aid in determining the scope of the claimed subject matter.

In accordance with an exemplary embodiment, an orthopedic system for conditioning a soft tissue structure is provided. The system comprises a distraction device, at least one sensor, and a controller. The distraction device includes a first connector configured to engage a first anatomical structure associated with a soft tissue structure, a second connector configured to engage a second anatomical structure associated with the soft tissue structure, and a displacement mechanism configured to apply a force to one of the first and second connectors for applying a tension to the soft tissue structure. The at least one sensor is configured to generate parameter measurement data associated with the soft issue structure and/or the distraction device upon the displacement mechanism applying the force to one of the first and second connectors, the parameter measurement data comprising at least one of a force, and a displacement of the soft tissue structure. The controller is operatively associated with the distraction device and the at least one sensor and configured to receive parameter measurement data from the at least one sensor, determine a characteristic parameter of the soft tissue structure based on the parameter measurement data, and control the displacement mechanism in response to the determined characteristic parameter until the determined characteristic parameter satisfies a predetermined target condition.

The anatomical structure is hard tissue or soft tissue, and the soft tissue structure is a graft, a ligament, a tendon, a muscle, cartilage, capsular tissue,meniscus, fascia, or connective tissue. The parameter measurement data is a force or a displacement, and the characteristic parameter is a tension force, a percentage elongation, a change in elongation, a length, an overall length, or a thickness. The distraction device is configured to engage the first and second anatomical structures in situ or ex situ. The controller is configured to control the displacement mechanism by updating the force applied to one of the first and second connectors. The controller is configured to control the displacement mechanism by applying a cyclical force to one of the first and second connectors. The cyclical force is applied over a predefined time period. The target condition comprises the characteristic parameter of the soft tissue structure having a delta measurement less than a predefined amount after successive updates to the control of the displacement mechanism. The predefined amount is 1-40% of the characteristic parameter. The controller is configured to receive parameter measurement data from the at least one sensor as the first and second anatomical structures are moved between first and second positions. The system further comprises a 3D tracking system configured to determine a position of the first and second anatomical structures. The controller determines a surgical plan based on the characteristic parameter at the target condition.

In accordance with another exemplary embodiment, a system for tensioning a soft tissue structure of a joint is provided. The system comprises a distraction device, at least one sensor, and a controller. The distraction device includes a first connector configured to engage a first anatomical structure associated with a soft tissue structure, a second connector configured to engage a second anatomical structure associated the soft tissue structure, and a displacement mechanism operable to displace the first connector relative to the second connector. The at least one sensor is configured to measure a parameter associated with the soft tissue structure and/or the distraction device, the parameter comprising at least one of a force, a tension, an elongation and a displacement of the soft tissue structure. The controller is operatively in communication with the distraction device and the at least one sensor. The controller is configured to: control the displacement mechanism to displace the first connector relative to the second connector upon engagement with the first and/or the second anatomical structures associated with the soft tissue structure, and apply a predetermined force to displace the first connector relative to the second connector, measure a tension of the soft tissue structure upon application of the predetermined force to displace the first connector relative to the second connector, determine a displacement height of the first and second connectors corresponding to the measured tension of the soft tissue structure reaching a predetermined tension, and measure a tension of the soft tissue structure as the soft tissue structure moves between first and second positions while the controller holds the displacement height of the first and second connectors at the determined height.

The system further comprises a navigation system to track a position of the first anatomical structure and the second anatomical structure. The system further comprises a display for displaying at least one of force data, tension data, elongation data and displacement data of the soft tissue structure relative to a position of the first and/or second anatomical structure.

In accordance with yet another exemplary embodiment, a system for measuring soft tissue structures of a joint is provided. The system comprises a distraction device, at least one sensor and a controller. The distraction device includes a first connector configured to engage a first bone of the joint or a soft tissue structure associated with the first bone of the joint, a second connector configured to engage a second bone of the joint or a soft tissue structure associated with the second bone of the joint, and a displacement mechanism operable to displace the first connector relative to the second connector. The at least one sensor is configured to measure a parameter associated with the soft tissue structure and/or the distraction device, the parameter comprising at least one of a force and a displacement of the soft tissue structure. The controller is operatively in communication with the distraction device and the at least one sensor. The controller is configured to control the displacement mechanism and apply a predetermined force to displace the first connector relative to the second connector of the distraction device upon engagement of the first and second connectors with the first and second bones or the soft tissue structure associated with the first bone or the second bone, determine a displacement between the first and second connectors and a degree of elongation of the soft tissue structure upon application of the predetermined force to the displacement mechanism, and display the degree of elongation of the soft tissue structure on a display.

The system further comprises a navigation system configured to track a position of the first bone, the second bone, and/or soft tissue structure. The at least one sensor is configured to measure at least one of force data, tension data, elongation data and/or displacement data of the soft tissue structure. The controller is further configured to display an elongation profile of the soft tissue structure as a function of joint position over a range of motion. The controller is further configured to quantify a laxity assessment of the joint while applying the predetermined force.

In accordance with another exemplary embodiment, a system for performing an orthopedic surgical procedure is provided. The system comprises a distraction device, at least one sensor, a user interface, a guide structure, and a controller. The distraction device is configured to apply a controlled force or displacement between first and second bones of a joint. The at least one sensor configured to measure at least one of force and displacement. The user interface is configured to display the measured force and/or displacement, and receive user input to control the applied controlled force and/or displacement. The guide structure coupled to the distraction device and configured to guide placement of a surgical instrument relative to the joint. The controller is operatively in communication with the distraction device, the at least one sensor and the user interface and configured to position the surgical instrument based on the measured force and/or the measured displacement applied by the distraction device.

The user interface is configured to selectively operate the distraction device in a force-control mode or in a displacement-control mode. The user interface is configured to enable independent adjustment of medial and lateral forces on the joint by the distraction device. The controller is configured to determine an implant size based on the measured displacement.

The foregoing general description of the illustrative exemplary embodiments and the following detailed description thereof are merely exemplary aspects of the teachings of this disclosure and are not restrictive.

### BRIEF DESCRIPTION OF THE DRAWINGS

The foregoing summary, as well as the following detailed description of the exemplary embodiments of the subject disclosure, will be better understood when read in conjunction with the appended drawings. For the purpose of illustrating the subject disclosure, there are shown in the drawings exemplary embodiments. It should be understood, however, that the exemplary embodiments are not limited to the precise arrangements and instrumentalities shown.

FIG. 1 is a perspective view of a computer-assisted orthopedic surgery (CAOS) system in accordance with an exemplary embodiment of the subject disclosure;

FIG. 2 is a schematic representation of a computer-assisted orthopedic surgery system in accordance with the subject disclosure;

FIG. 3a is a side view of an exemplary distraction device of the CAOS system of FIG. 1 as applied to a knee joint during an exemplary ligament reconstruction procedure;

FIG. 3b is a rear view of another exemplary distraction device of the CAOS system of FIG. 1 as applied to a knee joint during an exemplary ligament reconstruction procedure;

FIG. 3c is a side view of an exemplary distraction device of the CAOS system of FIG. 1 as applied to a knee joint during another exemplary ligament reconstruction procedure;

FIG. 4 is a side view of another exemplary distraction device of the CAOS system of FIG. 1 as applied to a knee joint during;

FIG. 5a is a perspective view of a distraction device in accordance with another exemplary embodiment of the subject disclosure;

FIG. 5b is a partial perspective of distal ends of connectors of the distraction device of FIG. 5a in an open configuration;

FIG. 6a is a distraction device in accordance with another exemplary embodiment of the subject disclosure in a closed configuration;

FIG. 6b is a side view of the distraction device of FIG. 6a in a closed configuration;

FIG. 7a is an exemplary output of the CAOS system of the subject disclosure represented as a graph of graft elongation vs. joint position;

FIG. 7b is another exemplary output of the CAOS system of the subject disclosure represented as a graph of graft tension vs. joint position;

FIG. 8 is a user interface display in accordance with an exemplary embodiment of the subject disclosure;

FIG. 9a is an exemplary output of the CAOS system of the subject disclosure represented as a graph of graft length vs. time;

FIG. 9b is an exemplary output of the CAOS system of the subject disclosure represented as a graph of tension vs. elongation;

FIG. 9c is an exemplary output of the CAOS system of the subject disclosure represented as a graph of graft length vs. time;

FIG. 10 illustrates a distraction device in accordance with another exemplary embodiment of the subject disclosure;

FIG. 11 illustrates a system diagram of a CAOS system in accordance with another exemplary embodiment of the subject disclosure;

FIGS. 12-15 illustrate various exemplary surgical workflows appliable with the CAOS system of the subject disclosure;

FIGS. 16-17 illustrate various exemplary configurations of the CAOS system applicable to knee arthroplasty procedures in accordance with the subject disclosure; and

FIGS. 18a-18b illustrate various femoral knee implants applicable for use in connection with the CAOS system of the present disclosure.

### DETAILED DESCRIPTION

Reference will now be made in detail to the preferred embodiments of the disclosure illustrated in the accompanying drawings. Wherever possible, the same or like reference numbers will be used throughout the drawings to refer to the same or like features. It should be noted that the drawings are in simplified form and are not drawn to precise scale. In reference to the disclosure herein, for purposes of convenience and clarity only, directional terms such as top, bottom, above, below and diagonal, are used with respect to the accompanying drawings. The term “proximal” refers to being nearer to the center of a body or a point of attachment. The term “distal” refers to being away from the center of a body or from a point of attachment. Such directional terms used in conjunction with the following description of the drawings should not be construed to limit the scope of the invention in any manner not explicitly set forth. Additionally, the term “a,” as used in the specification, means “at least one.” The terminology includes the words above specifically mentioned, derivatives thereof, and words of similar import.

“About” as used herein when referring to a measurable value such as an amount, a temporal duration, and the like, is meant to encompass variations of ±20%, ±10%, ±5%, ±1%, and ±0.1% from the specified value, as such variations are appropriate.

“Substantially” as used herein shall mean considerable in extent, largely but not wholly that which is specified, or an appropriate variation therefrom as is acceptable within the field of art. “Exemplary” as used herein shall mean serving as an example.

Throughout this disclosure, various aspects of the subject disclosure can be presented in a range format. It should be understood that the description in range format is merely for convenience and brevity and should not be construed as an inflexible limitation on the scope of the subject disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Furthermore, the described features, advantages and characteristics of the embodiments of the disclosure may be combined in any suitable manner in one or more embodiments. One skilled in the relevant art will recognize, in light of the description herein, that the invention can be practiced without one or more of the specific features or advantages of a particular embodiment. In other instances, additional features and advantages may be recognized in certain embodiments that may not be present in all embodiments of the invention. Accordingly, it should be recognized that the descriptions herein are not intended as a limitation on the scope of the present disclosure. Rather, the description also encompasses combinations and modifications to those exemplary aspects described herein.

In accordance with an exemplary embodiment, the present disclosure discloses a computer-assisted orthopedic surgery (CAOS) system 1000 and method for soft tissue assessment of a joint before and during orthopedic surgical procedures e.g., pre-operatively and intra-operatively. The system allows for measured and controlled tension and displacement of soft tissue structures associated with a joint, such as ligaments, tendons, grafts, joint capsules, muscle, cartilage, and fascia, before and during various surgical procedures. The system may include a distraction device 100 having connectors for engaging bones and/or soft tissue structures of a joint, a displacement mechanism for controlling the distance and/or force between the connectors, and a controller for applying predetermined forces and/or measuring distances.

The system 1000 may be applied to various joints throughout the body. For example, the system may be used in procedures involving a knee joint, a hip joint, an ankle joint, a shoulder joint, an elbow joint, a wrist joint, a spine, a temporomandibular joint, or metacarpophalangeal or metatarsophalangeal joints, and other similar joints.

The system may be used in various surgical procedures. for example, the system may be used in soft tissue reconstruction or repair procedures, such as ligament reconstruction procedures. In some cases, the system may be used in multi-ligament repairs involving the anterior cruciate ligament (ACL), the posterior cruciate ligament (PCL), and medial and lateral collateral ligaments of the knee. In some cases, the system may be used in procedures involving rotator cuff tendons around a shoulder joint. In other cases, the system may be used in hip and shoulder labrum tear repairs, such as superior labrum anterior to posterior (SLAP) repairs, associated ligaments such as the Iliofemoral ligament, Ischiofemoral ligament, and the pubofemoral ligament. The system may also be applied to arthroplasty procedures to optimize tension of soft tissues surrounding a joint during the arthroplasty procedure, such as procedures with the knee, hip, ankle, shoulder, elbow and spine.

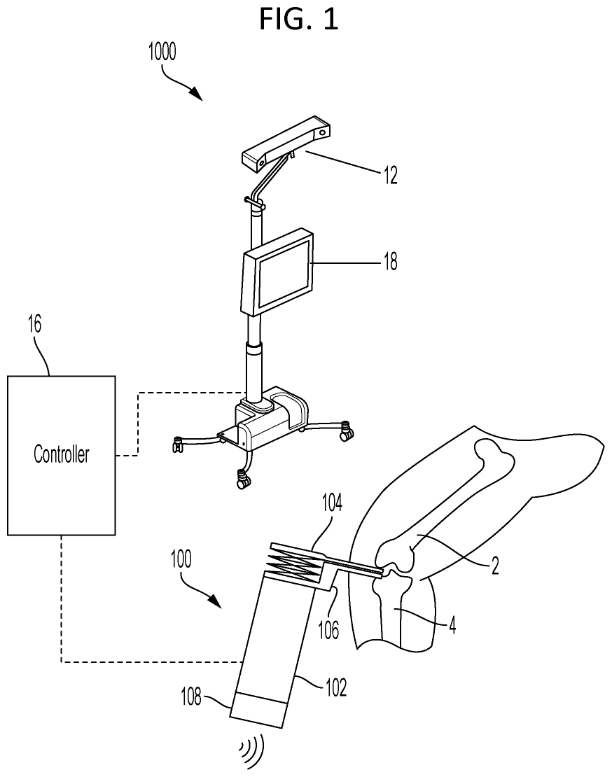

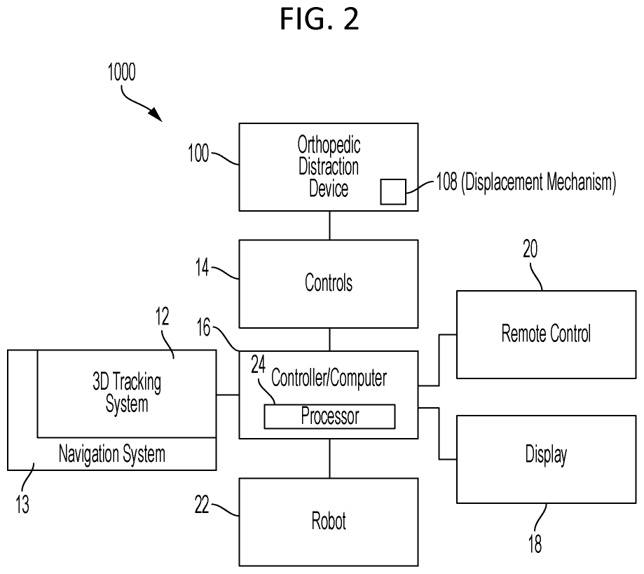

FIG. 1, illustrates a computer-assisted orthopedic surgery (CAOS) system 1000 according to an exemplary embodiment of the subject disclosure. The CAOS system 1000 is also referred to herein as an orthopedic system. FIG. 2 illustrates a block diagram of the CAOS system 1000. The CAOS system 1000 includes an orthopedic distraction device 100, a three-dimensional (3D) tracking system 12, user controls 14, a computer/controller 16, a display 18, a remote control 20, and a robot 22.

The 3D tracking system e.g., a navigation system, is configured to track position of bones of a joint, fiducial markers, and/or instruments (e.g., anatomy or point digitization probes, bone drills, drill guides, and bone cutting guides) in three dimensional (“3D”) space and relative to one another. The 3D tracking system is also configured to determine a position and/or track a position of the first and second anatomical structures. The 3D tracking system 12 may include any tracking technology, including optical, radiofrequency, radar, acoustic, markerless video-based, magnetic, radiographic, fluoroscopic, ultrasound, accelerometer, and gyroscopic tracking. Teachings directed to certain 3D tracking systems applicable to the computer-assisted orthopedic surgery system, can be found e.g., in U.S. Pat. Nos. 9,248,001, and 5,564,437, the entire disclosure of which are hereby incorporated by reference for all purposes.

The 3D tracking system can be a standalone system or part of a navigation system 13 having the 3D tracking system 12. Thus, the CAOS system can include a navigation system to track a position of the first anatomical structure and the second anatomical structure, a first bone a second bone and/or soft tissue structures, as well as all other objects discussed herein tracked by the 3D tracking system.

The computer 16 can include a memory and a processor 24 configured to process data and other inputs. The memory can include computer instructions executable by the processor, such that the processor is configured to perform various acts and/or functions, as further discussed in detail below. The computer and processor can collectively be referred to as a controller that is operatively in communication the orthopedic distraction device, the three-dimensional tracking system, the display, the remote control, and the robot.

The display 18 displays information to a user. The display 18 may present real-time data, graphs, and user interface elements to assist the surgeon during a procedure. Further, the display can display at least one of force data, tension data, elongation data, and displacement data of the soft tissue structure relative to a position of the first and/or second anatomical structures. A user may communicate with the system using the display through e.g., a touch screen interface. In some cases, the user may communicate with the system through a keyboard, mouse, voice control, gesture control, foot pedal, or the remote control 20.

The robot 22 can be a robot arm, an end effector and the like for positioning instruments such as drill guides, cutting guides, or tools for use during surgery. In some cases, the robot 22 may be used for manipulating objects, including bones of the joint, during surgery.

The various elements of the computer-assisted orthopedic surgery system are operatively connected to or in communication with one another. For example, the controller is operatively connected to or operatively in communication with the distraction device, the controls, the 3D tracking system, the display, the remote control and the robot.

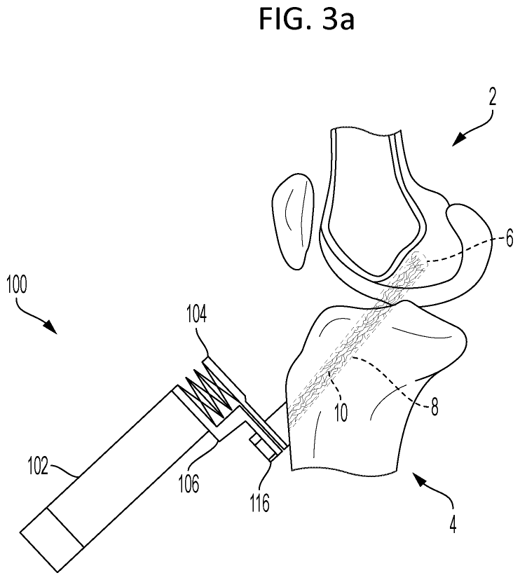

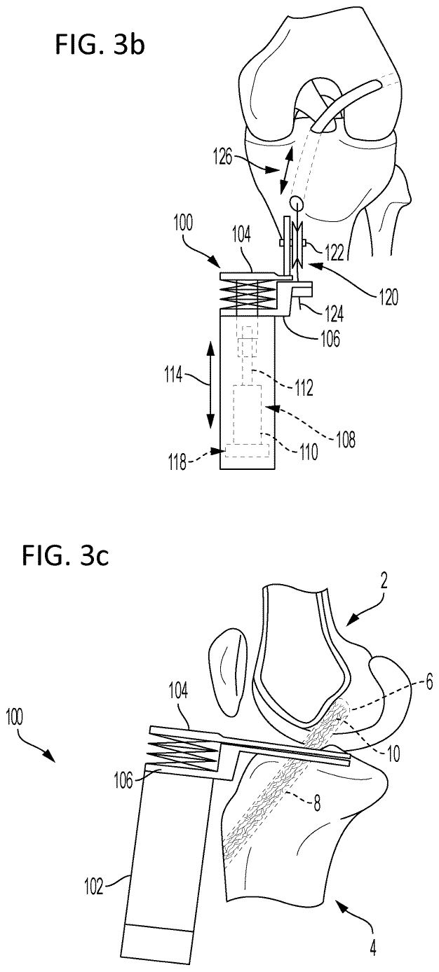

Referring to FIGS. 1 and 3a-c, the distraction device 100 includes a body 102, a first connector 104 and a second connector 106. The first and second connectors are diametrically opposed and movable relative to one another in opposite directions. The connectors can be configured as paddles, couplers or any other configuration suitable for its intended purposes and these terms may be used interchangeably and are broadly intended to represent physical members adapted to contact tissue such as hard tissue (bone) and soft tissue (cartilage, ligaments, tendons, joint capsule, muscle, biologic or synthetic grafts, and the like), for the purposes of applying forces or displacement between the tissues. Teachings directed to computer-assisted orthopedic surgery systems including exemplary distraction devices applicable to the subject disclosure can be found e.g., in U.S. Pat. No. 10,321,904, which is hereby incorporated by reference for all purposes in its entirety.

The distraction device 100 is configured for controlling a distance (height) and/or a force between the upper and lower connectors 104, 106 via the controller 16 and the displacement mechanism 108. The displacement mechanism 108 can include a drive assembly operable for controlling displacement and moving the first and second connectors between a first position and a second position spaced from the first position. The drive assembly may include motors, gears, encoders, air or fluid powered pistons or bladers, electromagnetic mechanisms, and the like, suitable for the intended purpose. In sum, the displacement mechanism is configured to apply a force to one of the first and second connectors. As such, the displacement mechanism can be used to apply a tension to a soft tissue structure, as further discussed below.

FIG. 3b illustrates an exemplary embodiment of a distraction device 100 in accordance with the subject disclosure. The distraction device includes a displacement mechanism or drive assembly 108 that includes a motor 110, a ball-screw drive 112 for transforming rotational motion of the motor 110 to linear motion (e.g., in a vertical direction 114) that is largely parallel to a longitudinal axis of the motor driveshaft. The ball-screw drive 112 is connected to one of the first (upper) connector 104 and the second (lower) connector 106 to allow the first connector to travel along the linear motion direction e.g., from a first position to a second position. The distraction device can also include a graft coupler 116 (FIG. 3a) for securing a soft tissue structure to the distraction device e.g., one of the first and second connectors. The distraction device is configured to engage the first and second anatomical structures in situ or ex situ.

A sensor 118 is included with the distraction device 100 for measuring e.g., the forces applied by the joint e.g., bone, soft tissue structures, and the like, to the first and/or second connectors along the direction of travel 114. Thus, the distraction device 100 via the sensor e.g., a force sensor, can measure the load applied to the upper and lower connectors 104, 106 and output the applied load data to the user via the display. The measured load can also be used to control a force applied by the distraction device 100 via the controller. The sensor is also configured to measure at least one of force data, tension data, elongation data and/or displacement date of soft tissue structures, as further discussed below. The sensor 118 can be positioned within or on the distraction device, such as in or on one or both connectors, or anywhere along or alongside or within the distraction device 100 e.g., the distraction device body, such that forces applied to the distraction device by contact with patient tissue can be measured.

In accordance with an exemplary embodiment, the subject disclosure provides an orthopedic system 1000 that includes a distraction device 100, at least one sensor 118, and a controller 16 operatively associated with the distraction device and at least one sensor. The distraction device 100 can include a first connector 104 configured to engage a first anatomical structure associated with a soft tissue structure, a second connector configured to engage a second anatomical structure associated with the soft tissue structure, and a displacement mechanism configured to apply a force to one of the first and second connectors for applying a tension to the soft tissue structure. The anatomical structure can be hard tissue or soft tissue and the soft tissue structure can be a graft, a ligament, a tendon, a muscle, cartilage, capsular tissue,meniscus, fascia, or connective tissue.

As used herein “soft tissue structure” means any soft tissue structure associated with a joint, including but not limited to, ligaments, tendons, muscles, cartilage, capsular tissue,meniscus, fascia, and connective tissue. The term “anatomical structure” as used herein means any structure including both hard and soft tissues. The term “anatomical structures associated with a soft tissue structure” means any anatomical structure connected to or operatively associated with the soft tissue structure, including portions of the soft tissue structure itself e.g., an end portion of a ligament can be considered an anatomical structure associated with said ligament, or a tendon extending from a ligament can be considered the anatomical structure associated with the ligament.

The sensor is operatively associated with the distraction device e.g., to sense or measure data from the distraction device, including forces applied by the distraction device, forces applied to the distraction device, and/or height or spacing of the first and second connectors e.g., upon engagement with soft tissue structures. The sensor is also configured to sense or measure i.e., generate parameter measurement data associated with the soft tissue structure and/or the distraction device upon the displacement mechanism applying forces to one of the first and second connectors when the displacement mechanism is operatively engaged during a surgical procedure. The parameter measurement data can be at least one of a force, and a displacement of the soft tissue structure e.g., change in position or relative position. The parameter measurement data can also include other mechanical properties of the soft tissue structure.

The controller 16 is operatively associated with the distraction device and the at least one sensor. The controller is configured to receive parameter measurement date from the at least one sensor, determine a characteristic parameter of the soft tissue structure based on the parameter measurement data, and control the displacement mechanism in response to the determined characteristic parameter until the determined characteristic parameter satisfies a predetermined target condition. That is, for example, the controller includes computer instructions stored in memory or downloadable into memory executable to receive parameter measurement date from the at least one sensor, determine a characteristic parameter of the soft tissue structure based on the parameter measurement data, and control the displacement mechanism in response to the determined characteristic parameter until the determined characteristic parameter satisfies a predetermined target condition. In other words, the controller is configured to receive parameter measurement data from the at least one sensor as the first and second anatomical structures are moved between first and second position. Additionally, the controller is configured to determine a surgical plan based on the characteristic parameter e.g., when the characteristic parameter is at the target condition.

The characteristic parameter of the soft tissue structure can be e.g., a percent elongation or percentage elongation, a delta or change in elongation, a length, an overall length, a thickness, a tension force, and the like.

The controller is configured to control the displacement mechanism in response to the determined characteristic parameter. That is, e.g., the controller can increase the force the displacement mechanism applies to the first and second connectors until the determined characteristic parameter satisfies a predetermined target condition. In other words, the controller is configured to control the displacement mechanism by updating the force applied to one of the first and second connectors. For example, the target condition can be a predetermined tension or maximum or a minimum tension force when the characteristic parameter is tension. When the characteristic parameter is percent elongation, the target condition can be a predetermined or predefined percentage change or delta of a length or an overall length of the soft tissue structure. The target condition can also be defined as the characteristic parameter of the soft tissue structure having a delta measurement less than a predefined amount after successive updates to the control of the displacement mechanism. The predefined amount applicable for the delta measurement can be 1, 2, 3, 4, 5, 10, 15, 20, 25, 30, 35, 40, 45 or 50% of the characteristic parameter or applicable ranges such as 1-50%, 1-40%, 10-40%, 20-30%, 1-30%, 1-20%, and 1-10% of the characteristic parameter.

The controller can also be configured to apply a constant force, and a distance/length can be monitored over time. A rate of change in length can be calculated as a characteristic parameter and monitored. The controller can also be configured to apply a varying force and rate of change can be calculated. A ratio between a rate of change in force and a rate of change of displacement/length can also be calculated as a characteristic parameter and monitored. The ratio can be expressed as Delta Force/Delta Length, or the inverse, Delta Length/Delta Force. This ratio can be calculated over a specific time period, for example 0.5 1, 2, 3, 4, 5, 6, 7, 8, 9, and 10 seconds or minutes.

The controller can also be configured to apply a controlled force to the displacement mechanism over a predefined period of time such that the displacement mechanism via the first and second connectors applies said force to the soft tissue structure over the predefined period of time. The predefined period of time can be 1, 5, 10, 15, 20, 25, 30, 45, or 60 seconds, or 1, 2, 3, 4, 5, 6, 7, 8, 9, 10 minutes.

The controller can also be configured to apply a controlled force to the displacement mechanism that varies over time such as a cyclic load profile in which the force increase over a predefined period of time until the force reaches a predetermined maximum or the force measured by the distraction device reaches a predetermined maximum. The predetermined maximum force can be e.g., 100-3,000 N for patellar tendon, 100-2,000 N for large joints of the human body e.g., knee joint, 5-300 N for smaller joints of the human body e.g., fingers and ankle. In other words, the controller is configured to control the displacement mechanism by applying a cyclic force to one of the first and second connectors.

The controller can also be configured to apply a controlled force to the displacement mechanism that varies over time such as a cyclic load profile in which the force increases over a predefined period of time until the percentage change or delta of a length of the soft tissue structure reaches a predetermined or predefined amount. The predetermined amount can be e.g., 1, 2, 3, 4, 5, 6, 7, 8, 9, 10, 15, 20, 30 or 40% by length or thickness. In other words, the cyclic force is applied over a predefined time period.

In accordance with another aspect, the distraction device is configured to apply varying distraction forces. That is, the distraction device's controller is configured to apply varying distraction forces to a graft (e.g., to a graft in a ligament reconstruction having one end attached to a bone of a joint and another end attached to the distraction device), based on a relative position of the bones of a joint. For example, applied forces of the distraction device may be automatically varied from a high force or higher force, when the knee is in an extended position, to a low force or lower force (e.g., a force lower in value than the high force) when the knee is in a flexed position. In this aspect, the joint position (flexion) can be measured with the 3D tracking system, and the force applied can be controlled with the controller, based on the joint position. Thus, a predetermined force profile can be applied to or obtained for the graft as a function of joint position.

In accordance with another aspect, the distraction device is configured to apply varying distraction height (connector separation distance). That is, the distraction device's controller is configured to apply varying distraction height to a graft (e.g., to a graft in a ligament reconstruction having one end attached to a bone of a joint and another end attached to the distraction device), to control graft elongation based on a relative position of the bones of a joint. For example, an applied height (i.e., distraction height) may be automatically varied from a high distance or higher distance, when the knee is in an extended position, to a low distance or lower distance (e.g., a low distance is a distance lower in value than the high distance), when the knee is in a flexed position. In this aspect, joint position (flexion) can be measured with the 3D tracking system, and the distraction device height applied can be controlled with the controller based on joint position. Thus, a predetermined height, or ligament elongation profile, can be applied to or obtained for the graft as a function of joint position. In other words, the controller is configured to display an elongation profile of the soft tissue structure as a function of joint position over a range of motion.

FIGS. 3a and 3b illustrate an exemplary application of the orthopedic system 1000 to an exemplary ligament reconstruction procedure e.g., an anterior cruciate ligament reconstruction. FIG. 3a is a lateral view of a femur 2 and a tibia 4 of a knee joint undergoing an arthroscopic ACL. Tunnels 6 and 8 have been drilled in the femur 2 and tibia 4, respectively, to accept graft 10. Tunnels 6 and 8 may have been positioned using surgical navigation, a 3D tracking system, and graft isometry/anisometry and impingement data, for example, as described in U.S. Pat. No. 9,248,001 (incorporated by reference above).

The graft 10 can be attached (anchored) to the femur 2 through a femoral tunnel 6 which enters an inside of the joint around an area of the femoral notch. The graft 10 may be anchored in the tunnel by any manner known in the art, such as sutures, sutures and a button, staples, interference screw, cross-pins/screws, bone anchors, biologic fixation, and the like. An opposite end of the graft 10 is passed through the tibial tunnel 8 and attached to the lower or second connector 106 of the distraction device 100 via a graft coupler 116. The graft coupler 116 can be adapted to attach to the graft 10 by various methods, such as with sutures, sutures and a suture button (through which the sutures pass through), a vice for gripping the graft directly, a clamp, an anchor, and the like. The upper or first connector 104 may be configured to allow the graft 10 to pass through to the graft coupler 116 unobstructed. For example, the upper connector can be configured as a U-shaped connector. The upper connector 104 is also adapted to or structured to contact and/or be fixed to the tibial bone 4 to allow transfer of an opposing force to the tibial bone. Thus tibial fixation can be achieved through bone screws, pins, or contact surfaces that maintain the distraction device in place.

Referring to FIG. 3b, the distraction device may optionally include a pulley 120 that extends from one of the first and second connectors and rotates about rotational axis 122 for redirecting the direction of the travel or tension of the graft and/or graft attachment sutures 124. The pulley includes low friction curved guide surfaces for directing or engaging or receiving the graft thereby providing for freedom of positioning, and/or movement of a line of action 114 of the distraction device 100 with respect to the graft or graft tunnel direction 126. This arrangement allows the distraction device 100 to be positioned or repositioned, or fixed to the tibia, in any position without causing the graft to rub against, engage or contact the tibial bone or an opening of the tibial bone tunnel 8. For example, the distraction device 100 can be held by hand while being placed in contact with the bone, and allowed to pivot about certain contact points. This allows the distraction device 100 to apply a tension to, and to measure the tension of, the graft more accurately without significant loss due to friction or engagement with bone or a tunnel bored into the bone associated with a ligament reconstruction. Alternatively to the pully 120, the distraction device 100 can include straps, fasteners, pins, and/or a leg holder in which the tibia is secured for securing to the anterior portion of the tibia (or bone) or shank of a bone.

FIG. 3c illustrates another exemplary aspect of the distraction device of an orthopedic system in accordance with the subject disclosure where the distraction device 100 is operable to contact and distract a first bone from a second bone of a joint. The exemplary joint shown in FIG. 3c is a knee joint having a femur and tibia. The distraction device operates to distract the first and second bones of the joint by placing the first (upper) and second (lower) connectors (or couplers or paddles) between the first and second bones of the joint and controlling the distraction device to apply a force to displace one of the first connector and second connector from the other of the first connector and the second connector.

With the distraction device so configured between first and second bones of a joint, the orthopedic system's controller can apply forces to the distraction device to displace the first bone from the second bone of the joint and determine or measure a force applied to the first and second connectors of the joint distraction device or a distance or height of the first connector from the second connector. The forces applied to the distraction device's first and second connector by the first and second bones can then be used to determine a tension force of the ligaments associated with the joint e.g., the medial and lateral collateral ligaments of a knee joint. The measured tension forces are representative of the tension forces existing on the ligaments of the joint. The distraction device can then be used to move the joint bones to a target position such that the medial and lateral collateral ligaments exhibit a predetermined tension force (e.g., an optimized tension force) and determine a position or height of the distraction devices first and second connectors. The determined position or height of the first and second connectors and/or tension forces on the joint ligaments (collectively joint parameter data) can then be used by the controller to determine or update a surgical plan for an arthroplasty or soft tissue procedure associated with the joint. For example, the joint parameter data can be used to optimize depth of bone cuts (e.g., a tibial resection cut, distal femur cut, posterior femoral cut, and the like), implant space thicknesses, joint implant bearing thicknesses and the like. Similarly, the distraction device can be used for other joints and other joint parameter data associated with e.g., the hip joint, ankle joint, shoulder, elbow, and spine.

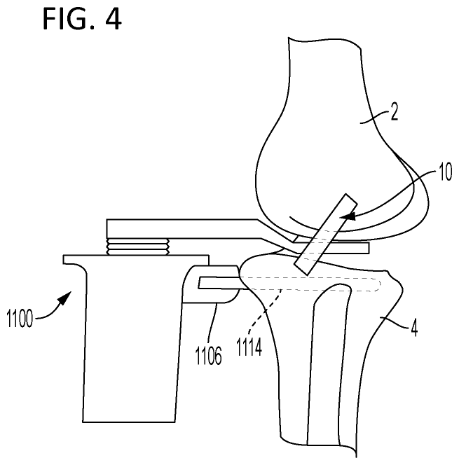

FIG. 4 illustrates another exemplary embodiment of an orthopedic system in accordance with the subject disclosure where the distraction device 100 is operable to affix the second (i.e., the lower) connector 1106 (or coupler or paddle) to an outside surface of a joint bone or from a direction facing an outside surface of a joint bone. For example, the lower connector can be affixed to an anterior surface of a tibia bone of a knee joint via pins 1114, screws, and the like.

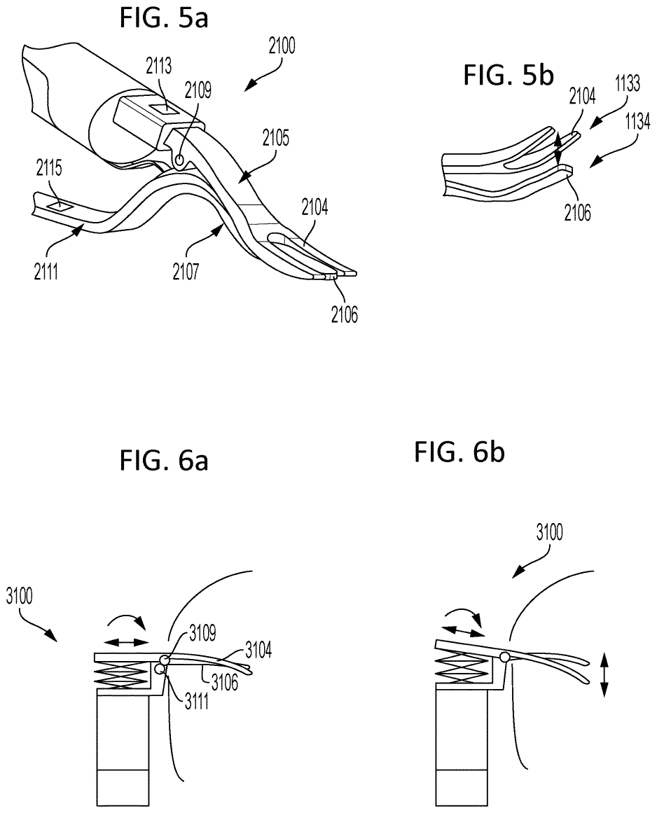

FIGS. 5a and 5b illustrate another exemplary embodiment of a distraction device 2100 having an upper connector/paddle 2104 and a lower paddle/connector 2106. The upper and lower paddles are configured as best shown in FIGS. 5a and 5b so as to have a low profile design for fitting into tighter spaces or small spaces. In the exemplary embodiment shown in FIG. 5a, the upper paddle is configured as a fork have a plurality of tines e.g., two tines and a curved longitudinal profile for complementing a curve provide of a bone's surface. The lower paddle is configured as a fork having one or more tines. The tines of the upper and lower paddles can be tapered such that the overall thicknesses of the tines decreases towards its distal end or tip. In the present exemplary embodiment, the lower paddle includes a single tine sized and shaped to be received between the two tines of the upper paddle and similarly curved in shape to have a curved longitudinal profile that matches the upper paddle when it inter lies with the upper paddle. So constructed, when the upper paddles inter lie with the lower paddles, the overall height of the tines of both the upper and lower paddles is that of a single tine of either of the upper or lower paddles.

The upper paddle extends from a first arm or upper arm 2105 and the lower paddle extends from a second arm or lower arm 2107. The lower arm is also configured to have a longitudinal extent or profile having a point of inflection spaced from its attachment point to the upper paddle. When in the closed position (FIG. 5a), the paddles 2104, 2106 are in a low profile configuration having an overall height equal to or substantially equal to an overall height a single tine. The overall height of a tine can be e.g., 1, 1.2, 1.4, 1.6, 1.8, 2, 3, 4, 5, 6, 7, 8, 9, or 10 mm. The curved profile of the upper and lower paddles advantageously allows them to better conform to surrounding joint or tissue anatomy, such as a femoral-tibial articulation or morphology of a knee joint or glenoid-humeral articulation or morphology of a shoulder joint.

The distraction device 2100 also includes a pivot 2109, about which the arms 2105, 2107 can pivot, and may include a handle or lever 2106 that can be used for manually displacing the upper 2104 and the lower 2106 paddles from one another. In other words, the lower paddle can be pivotably connected to the upper paddle so as to allow the distal ends of the upper and lower paddle tines to move between first and second positions such as a closed position (FIG. 5a) and an open position (FIG. 5b).A force sensor 2113 may be incorporated to measure a force acting between the upper 2104 and the lower 2106 paddles (e.g., a strain gage force sensor which is responsive to a deformation of a surface to which it is adhered; and through a calibration procedure that transforms the deformation to a force that is acting near the tip of the upper and/or lower paddles), as well as a position sensor 2115 to measure a separation of, or height between, the upper and lower paddles. The handle 2111 can be manually actuated or moved to open the upper and lower paddles (as shown in FIG. 5b) to create a separation distance between the two paddles, and the distraction device 2100 can monitor the displacement (or height or opening) at (between) the tip of the paddles, as well as the force acting on one or both of the paddles, or between the two paddles.

FIGS. 6a and 6b illustrate another exemplary distraction device 3100 operated through a small opening, such as a portal made in the patient body, to access a joint during an arthroscopic procedure or a mini-open procedure. In this aspect, owing to the pivot joint configuration (similar that shown in FIG. 5a) there is minimal expansion of the upper and the lower arms 3104, 3106 upon insertion through the portal. Upon insertion of the distraction device arms, the pivot joint 3109 is positioned in or near or adjacent the portal entry. This arrangement allows the distraction device 3100 to fit through a small portal and remain compact sufficient for passage through the portal area while being able to expand and distract at the paddle tips (which can be inside the patient body or joint), as shown in FIG. 6b. An articulating joint 3111 may also be incorporated between the upper paddle 3104 and the distraction device 3100 to allow for additional degrees of freedom, such as translation and angulation, to accommodate the pivoting motion of the upper paddle 3104 relative to the lower paddle 3106 about the fulcrum 3109.

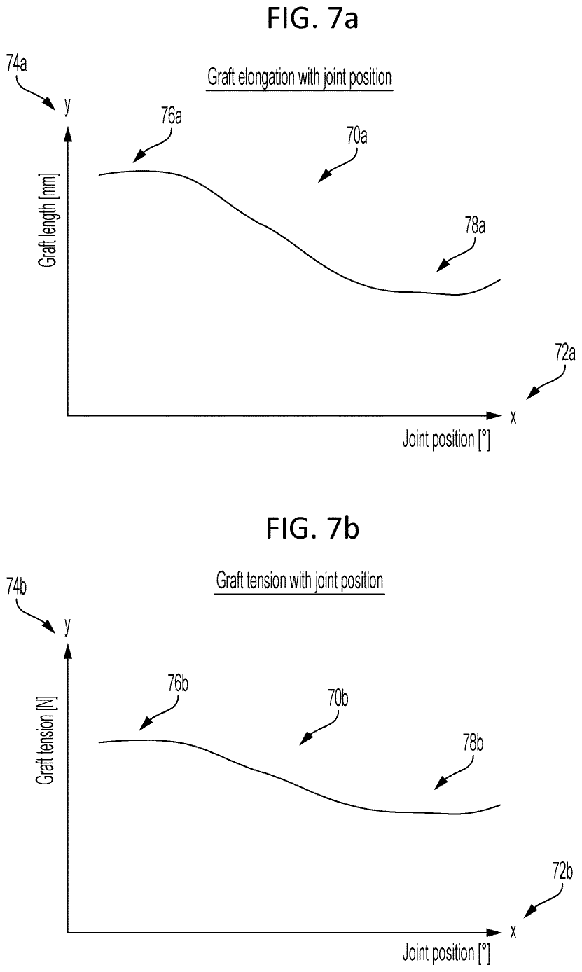

FIG. 7a illustrates an example of a graph of ligament elongation profile curve 70a that can be obtained by the distraction device of the subject disclosure applying a controlled load to a graft or other soft tissue structure used in a ligament reconstruction procedure as a surgeon takes a joint for which the ligament reconstruction procedure is applied to through a range of motion. The ligament elongation profile curve can be shown or displayed on the display 18 of the CAOS system. The graph includes an x axis 72a representing joint position (e.g., in degrees of flexion or mm of displacement) and a y axis 74a representing ligament elongation (e.g., in mm). Elongation can refer to a measurement of ligament length, or a change in ligament length, from an initial reference length, such as an initial length at a reference joint position (e.g., full extension or 30 degrees of flexion), or at a resting length when the ligament has negligible or low tension acting on it.

As can be seen in the elongation profile curve, the graft has an initial length 76a which is relatively long at the start of the x axis (extension position), indicating the ligament is longer when the joint is in this position. The elongation profile curve indicates that the graft length progressively decreases 78a as the knee is flexed, indicating that a lower ligament distance is required to maintain the applied tension in the ligament. Therefore, the ligament elongation profile curve provides an indication of the degree of isometry (or anisometry) of the ligament (i.e., the amount of change in ligament length) while the distraction device is applying a controlled load to the graft as a surgeon takes the joint through a range of motion.

Alternatively, a robot (such a robot 22) can be utilized to assess ligament stability of a patient's joint in combination with the distraction device. In this aspect, the robot is configured to move a patient's joint through a range of motion, while the distraction device is operatively situated between first and second bones of the patient's joint. For example, the robot can be utilized to secure a graft or ligament end to a first bone (e.g., a femur), and move the first bone relative to a second bone (e.g., a tibia) through an arc or range of motion.

FIG. 7b illustrates an example of a graph of ligament tension profile curve 70b that can be obtained by the distraction device of the subject disclosure applying a controlled load to a graft or other soft tissue structure used in a ligament reconstruction procedure as a surgeon takes a joint for which the ligament reconstruction procedure is applied to through a range of motion. The graph can be shown on the display 18 of the CAOS system with the x axis 72b representing a joint position (e.g., in degrees of flexion or mm of displacement) and the y axis 74b representing ligament tension (e.g., in Newtons, N). To measure the tension of a graft (used in a ligament reconstruction or repair) through different relative positions of the joint, a surgeon can first set the distraction device to apply a set or predetermined or initial tension to the graft while the joint is in a first or initial or predetermined position, after one end of the graft has been fixed to one of the bones of a joint, for example, a femur of a knee joint. The graft can be attached to the lower arm of the distraction device and the distraction device can apply the set load. In this aspect, once the initial tension is achieved, the distraction device may be locked into position by controlling the motors in the distraction device to maintain their position (i.e., controlling the height of the paddles/connectors) thereby fixing the length of the ligament relative to the tibia. The force sensor in the distraction device can then measure the tension in the ligament or determine tension based on measured forces from the force sensor, which can change as the joint is moved through a range of motion. Thus, the system can be used to measure and record a change in ligament or graft tension as a joint is moved through a range of motion.

As shown in the ligament tension profile curve, the graft has an initial tension 76b which is relatively high at the start of the x axis (extension position), indicating the ligament is under higher force when the joint is in this position. The tension profile curve illustrates that the tension progressively decreases 78b as the knee is flexed, indicating that the ligament is under less tension in flexed positions of the joint. Therefore, the ligament tension profile curve provides an indication of the degree of change in tension of the ligament while the ligament length is fixed, or controlled by the distraction device, as the surgeon takes the joint through a range of motion.

In accordance with another aspect, the CAOS system can include a 3D computer model of a joint that includes a shape model of the femur 80 and the tibia 82, as well as a computer model of the ligament 84. Because the bones and tunnel locations can be tracked in 3D, the impingement of the graft with the bone can be determined for various relative positions of the joint. The ligament can be modeled as a simple straight line, or as a cylinder with a diameter representing the diameter of the ligament graft.

A degree of impingement can be calculated based on a degree of intersection of the ligament model and the bone surface model. The ligament model can also be a bendable (non-linear) model to simulate a ligament wrapping around a bone surface when in contact with the bone. This aspect allows an elongation value to be calculated based on an amount of wrapping of the ligament model around the bone surface. In other words, the controller can determine elongation or a change in elongation based on an amount of wrapping of the ligament model around a bone surface of the bone model i.e., ligament impingement data. Thus, the CAOS system has a controller configured to determine: 1) whether or not the ligament is impinging against part of a bone, e.g., the roof of the notch of the femur; and/or a degree of impingement based on the relative positions of the bones. The ligament tension and elongation data measured with the distraction device can be combined with ligament impingement data, and outputted on the display of the CAOS system.

In accordance with another aspect, the CAOS system includes a controller configured to measure joint kinematics and laxity during various joint stability assessment motions e.g., joint stability exams such as an anterior-posterior (AP) drawer test, Lachman test, a pivot shift test, and a varus-valgus stability test. By tracking the kinematics of certain anatomical landmark points during the joint stability exams, a distance value can be calculated to represent a degree of laxity of the joint. For example, one or more points on a first bone of the joint e.g., a tibia of the knee joint, can be tracked with respect to a second bone of a joint e.g., a femur of the knee joint, during the assessment, and their displacement in a specific direction or degree of freedom can be measured, determined and reported.

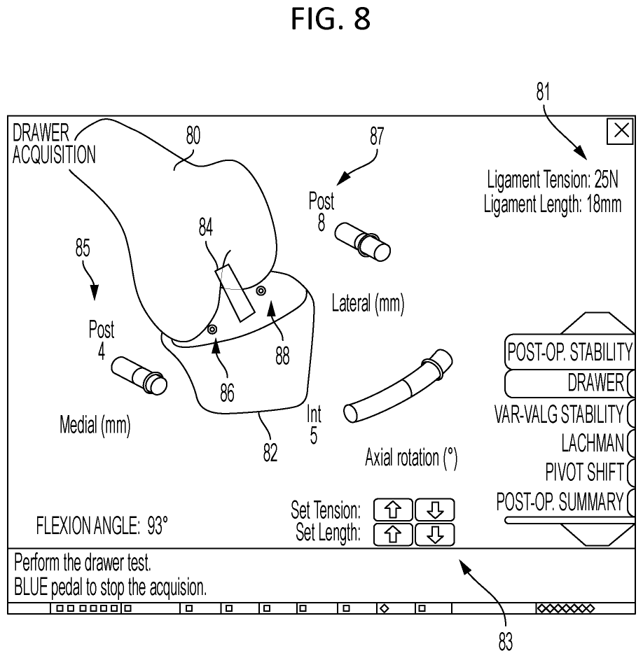

For example, FIG. 8 illustrates that displacement of a medial 86 and/or a lateral 88 tibial plateau point in the anterior direction of the tibia which can be measured and reported during an AP drawer test, when a knee is positioned at approximately 90 degrees of flexion and the tibia is pulled relative to the femur in an anterior direction of the tibia. Teachings directed to laxity tests can be found e.g., in U.S. Pat. No. 9,248,001 (incorporated by reference above).

In this aspect, joint laxity tests can be performed before and/or after graft fixation, and while the distraction device is controlling a force (tension) on the graft, or while the distraction device is controlling a length of the graft and measuring tension on the graft 84. Thus, a degree of joint laxity can be assessed while the distraction device is holding the graft at a set, initial, or predefined tension (or at a set length) which may be a predetermined tension (or a predetermined length). Furthermore, the tension of the graft can be varied by the distraction device, for example, by using a touch screen interface 83 (as shown in FIG. 8) to control the applied force, while a surgeon assesses joint laxity, where the degree of laxity can be assessed for various tensions applied to the graft. Thus, the CAOS system provides a controller configured to quantify a laxity assessment of the joint while applying a predetermined force.

In this aspect, a surgeon can find or determine an optimal tension for a graft by controlling the tension in the graft and assessing the joint laxity until a predetermined target condition of a degree of laxity is attained, or until a predetermined target condition of a combination of laxity and graft tension is attained. For example, during an initial AP drawer test, a surgeon might evaluate a joint with graft tension set with 20N of tension and locked (i.e., the tension can be set and locked in extension, or any degree of flexion, such as 90 degrees or 15-160 degrees). With the graft fixed at a set length, a surgeon can position the knee to 90 degrees, while monitoring a degree of tension in the graft, which may be displayed in real time on the screen display. The surgeon can then conduct an AP drawer test while assessing the readings for joint laxity 85, 87 and ligament tension 81. For example, the display may show an AP drawer laxity reading of 10 mm and a graft tension of 20N. The surgeon can then assess if this is too much laxity and not sufficient graft tension, and as such increase the degree of tension in the graft with the distraction device, using touch screen interface buttons 83 on the touch screen display (FIG. 8). The surgeon can then repeat the AP drawer test and re-evaluate the updated readings, which may now display e.g., a laxity of 5 mm and a graft tension of 40N. The display may show the graft tension and joint laxity values in real time, as well as a maximum tension and laxity value achieved during the joint laxity assessment.

Additionally, a laxity parameter and graft tension or elongation parameter may be displayed on a graph, such as those shown in FIGS. 7a and 7b. In this aspect, the x axis (joint position) represents the laxity parameter, such as AP translation in mm; and the y axis represents graft tension in N, as measured during the drawer test. In this aspect, one might expect to see graft tension increase as AP translation increases, and the curve move upward. If the curve is flat, that may indicate that tension in the graft is not increasing with increased translation and the surgeon may decide to increase the tension of the graft to increase the stability of the joint and to reduce the laxity, or change the position of the graft and or graft tunnels in the joint to achieve a different characteristic laxity-tension, or tension-elongation, or laxity-elongation profile. In other words, the user or surgeon can update a surgical plan for the joint or graft based on the data obtained from the joint distraction device, such as a graph of joint position v. graft length or joint position v. graft tension.

The system is also configured to display (on the display) and record (for example in computer memory) the preoperative (pre graft fixation) and postoperative (post graft fixation) laxity and ligament assessment data, such that a comparison of preoperative and postoperative data can be made and documented. The data may be presented side-by-side to easily allow for comparison. Further, such data can be used by the user or surgeon to update a surgical plan regarding the joint or graft.

### Preconditioning/Conditioning Tissue Properties

The CAOS system advantageously provides a means to precondition or condition soft tissue structures, either prior to or during implantation, or during repair of the soft tissue structure. The CAOS system can precondition ligaments and grafts to minimize time (history) dependent properties of the tissue, such as creep (i.e., deformation of tissue over time under constant stress) or stress relaxation (i.e., decrease in stress of tissue over time under constant strain). The CAOS system via its controller can be programmed or include computer instructions executable so that the distraction device can provide controlled cyclical loading, or cyclical displacement, to a soft tissue structure. The CAOS system then records and determines a change in mechanical properties of the soft tissue structure, such as tissue elongation or strain (or, in the case of application of controlled displacement, monitor tissue stress or force) over time and over each cycle. Such mechanical properties can be outputted to a display.

In one aspect, the pre-conditioning/conditioning can be done with the graft in situ, e.g., where one end of the graft is fixed to a first bone of a joint e.g., a femur, and the other end of the graft is attached to the distraction device's lower connector. The distraction device can then be set to apply a fixed or constant cyclical load or a varying force cyclical load between a certain minimum and maximum load, for example, between 100-2,000 N,100-500N, 20-120 N, and 0-80 N.

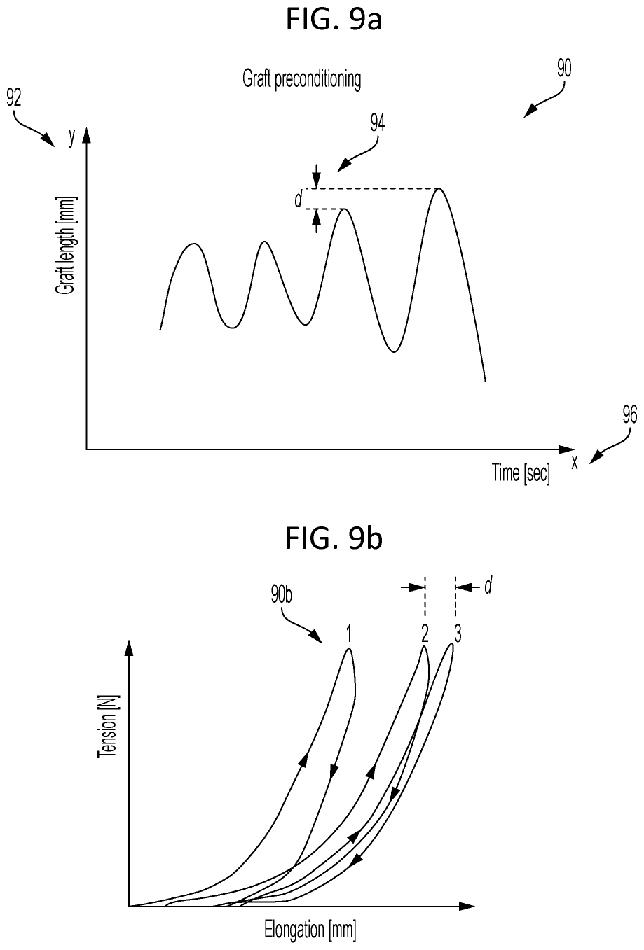

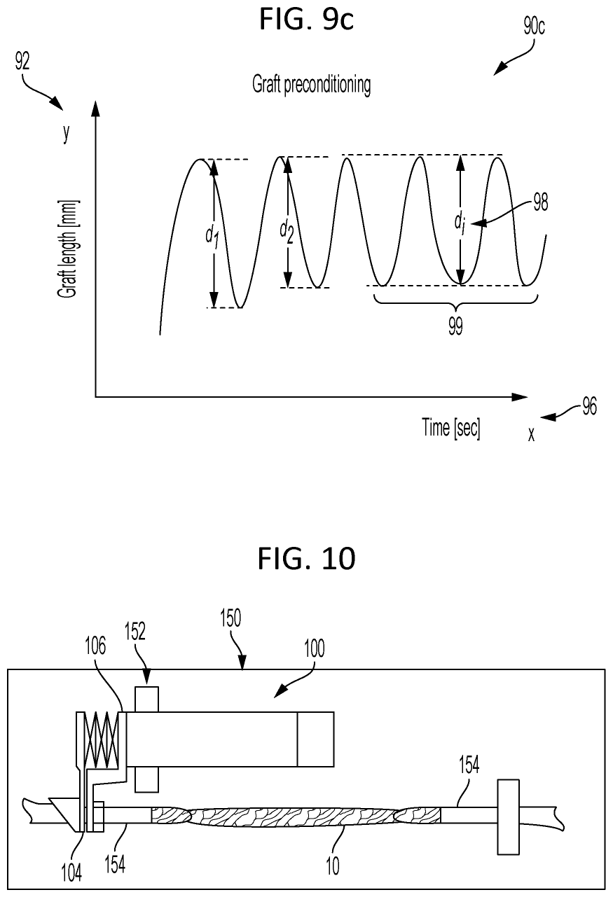

As shown in FIG. 9a, the CAOS system can measure and plot 90 a graft length 92 (y axis) v. time 96 (x axis) while applying a cyclical load waveform. As more load cycles are applied, the amount of tissue displacement (delta) may change, for example, an amount of displacement may increase, indicating some creep has occurred in the material. In this aspect, the increase in length, with each load cycle, may be measured (as parameter measurement data) and calculated or determined (as a characteristic parameter e.g., parameter d 94), which is determined as a difference in maximum elongation or length between different cycles of loading). Parameter d may be monitored over time, or with each cycle count. Predetermined target conditions can be established to determine a degree of preconditioning desired, and when sufficient preconditioning has occurred, for example, a stabilization of one or more characteristic parameters or parameter measurement data monitored during preconditioning. For example, when a percentage change in parameter d reaches a sufficiently small amount (e.g., <1%, 5%, 10%, 15%, or 30%) over a certain number of cycles (e.g., 1, 2, 3, 4, 5, 6, 7, 8, 9, 10, 15, or 20), and after a certain minimum number of overall cycles have been reached (e.g., after 5, 10, 25, 50, 75, or 100 cycles), or after a certain time period (e.g., 1, 5, 10, 20, 30, 40, 50, 60 secs or 2, 3, 4, 5, 6, 7, 8, 9, or 10 mins).

FIG. 9c shows another example of a graft length vs time graph 90c (with x-axis 96 in seconds and y-axis 92 mm), where a cyclical load is applied by the distraction device and the amount of displacement or change in a soft tissue structure length is measured, and a characteristic parameter corresponding to an amplitude of a graft length 98 is measured for each loading cycle (denoted d₁, d₂, . . . dᵢ). A region of stability 99 can be defined when the change in graft length (dᵢ) reaches a stable value, for example within a threshold value e.g., 0.1, 0.5, 1, 1.5, 2, 3, 4, 5 mm or more).

A speed of cyclic loading applied by the controller via the connectors may also be varied or tuned (e.g., 1, 2, 3, 4, 5, 6, 7, 8, 9, or 10 Hz), for example, to achieve tissue stabilization in a quicker time period (for instance, when preconditioning tissue while in-situ, versus preconditioning before inserting a graft, for example, during graft preparation on a back table) (FIG. 10). Moreover, in certain aspects, these parameters (e.g., applied force, distance, rate of loading or cycles per second, shape of load or displacement application waveforms (e.g., sinusoidal, saw tooth, square, rectangular, triangular, etc.)) may be optimized for different requirements. In one aspect, different requirements may be directed to different graft materials (e.g., bone-tendon-bone grafts, anterior tibialis, semitendinosus/gracilis, etc.) which respond differently to different preconditioning parameters, or time requirements. For example, if a ligament is preconditioned in situ (while in the joint), it may be desirable to minimize preconditioning time to not delay the surgery.

Soft tissue structures may be preconditioned before insertion into a joint, for example, as shown in FIG. 10 using a back-table setup. In this aspect, more time may be available to precondition a ligament, and a surgeon may choose to set preconditioning parameters that run over a longer time period. Such pre-operative preconditioning may be performed on a table setup, configured as best shown in FIG. 10. The table setup may include a board 150, a bracket or cradle 152 for securing the distraction device 100, and components for securing the graft 10 (which may have fixation sutures 154 on either end) to the board 150, and to the distraction device 100, to either the upper 104 connector/coupler or the lower connector/coupler 106. The board 150 may also include a measurement gauge or sensor for measuring graft length, and features such as holding pegs to assist with holding the graft while attaching sutures to the graft.

The foregoing may be used to plot the preconditioning data e.g., tension vs elongation, or a deformation plot 90b, as shown in FIG. 9b. In this example, FIG. 9b shows a ligament tension-elongation relationship over three progressive cycles (labelled 1, 2 and 3). Arrows on the curves indicate direction of curve progression over time with each cycle. In this aspect, a characteristic parameter d may also be calculated as a difference in elongation between cycles, as shown.

Preconditioning can also be performed using a constant controlled load applied by the distraction device over time. Graft length can be monitored over time and a characteristic parameter can be calculated (such as rate of elongation with time), and monitored, and used to inform a surgeon when sufficient preconditioning has occurred e.g., when the characteristic parameter satisfies a predetermined target condition. Alternatively, preconditioning can be performed by applying a controlled displacement (e.g., 5 mm) with the distraction device, or an amount of displacement to achieve a certain amount or increase in tension (e.g., 80N of tension), and then the height of the displacement device can be fixed and tension in the graft can be monitored over time until certain criteria have been met, at which point a user can be informed via the display, an audible cue, or the like. In this aspect, a decrease in tension can be measured over time, and when the tension has sufficiently stabilized (e.g., reached a threshold level (e.g., 60N)), the CAOS system can indicate to a user that preconditioning is complete and the graft is ready for implantation.

A quality metric of graft preconditioning can be determined and be used to indicate sufficient preconditioning of grafts, which can prevent a graft from loosening, or becoming too lax in the joint, and therefore not providing sufficient joint stability postoperatively, which may lead to premature failure, possibly requiring another subsequent operation or intervention. Such quality metric of graft preconditioning can be used to update a surgical plan for a graft or procedure on a joint. Exemplary quality metrics can be a metric indicative of the variation (such as the average variation, average absolute variation, standard deviation (often denoted by the Greek letter sigma), or variance (average squared deviation, or sigma squared)) of the amplitude of the graph length dᵢ over a certain number of cycles or over time, or over the region of stability 99 as shown in FIG. 9c. The quality metric could be transformed to a normalized scale from 0-1 or 0-100%, where a number closer to 0 indicates poor quality (for example high variation) and a value close to 100% indicates high quality. For instance, the variance could be normalized by the equation 1−sigma/dmax, where sigma is the variance and dmax is the maximum amplitude measured.

### CAOS System Workflows

The CAOS system and various embodiments associated therewith of the subject disclosure provide for various workflows e.g., of ligament reconstruction/repair procedures. Non-limiting examples are described below and such workflows may include some or all of the listed steps, in non-restrictive order, which may be accomplished with the CAOS systems and methods discussed above.

Workflow 1: Soft tissue structure elongation measured through a range of motion.
a) preconditioning a graft until a preconditioning predetermined target condition is satisfied;
b) creating a tunnel in a first and a second bone of a joint to receive a graft, where a position of the tunnels are determined via a navigation system tacking the first and second bones of the joint via a 3D tracking system;
c) creating 3D computer models of the first and second bones of the joint and said tunnels in the first and second bones;
d) conducting a laxity assessment of the joint to measure and record a baseline laxity assessment data set of the joint;
e) attaching a first end of the graft to the first bone;
f) attaching an upper connector of a distraction device of the CAOS system to the second bone;
g) attaching a second end of the graft to a lower connector of the distraction device, such that the distraction device can apply an opposing load to the graft and the second bone;
h) placing the joint at a first flexion angle and tracking the flexion angle via the 3D tracking system;
i) setting a target load for the distraction device to apply to the joint and/or graft;
j) applying the target load with the distraction device to create and control a tension in the graft;
k) moving the joint through a range of motion or through a laxity assessment motion while measuring joint position;
l) measuring elongation of the graft while applying the load with the distraction device or the 3D tracking system;
m) plotting an elongation profile vs joint position (e.g., flexion and/or AP translation during the AP drawer test); and
n) adjusting the tension in the graft using a human-machine interface, and repeating steps (h) through (m), until at least one of a joint stability, ligament tension and/or ligament elongation satisfies the predetermined target condition.

Workflow 2: Tension measured through a range of motion.
a) preconditioning a graft until a preconditioning predetermined target condition is satisfied;
b) creating a tunnel in a first bone and a second bone of a joint to receive the preconditioned graft, where a position of the tunnels are determined via a navigation system tacking the first and second bones of the joint via a 3D tracking system;
c) creating 3D computer models of the first and second bones of the joint and said tunnels in the first and second bones;
d) conducting a laxity assessment of the joint (e.g., an AP drawer test), to measure and record a baseline laxity assessment data set of the joint;
e) attaching a first end of the graft to the first bone;
f) attaching an upper connector of a distraction device to the second bone;
g) attaching a second end of the graft to a lower connector of the distraction device, such that the distraction device can apply an opposing load to the graft and the second bone;
h) placing the joint at a first flexion angle, and tracking the flexion angle via the 3D tracking system;
i) applying a load with the distraction device to create tension in the graft;
j) locking (holding) the distraction device height to maintain the graft in said tension while at the first flexion angle;
k) moving the joint through a range of motion while tracking the joint position;
l) measuring tension (force) in the graft using a sensor associated with the distraction device, while holding/maintaining the distraction device height;
m) plotting a tension profile vs joint position (e.g., flexion or translation during AP drawer test); and
n) adjusting the tension in the graft using a human-machine interface, and repeating steps (h) through (m), until at least one of a joint stability, ligament tension and/or ligament elongation satisfies the predetermined target condition.

Certain non-limiting variations to the above CAOS system's descriptions and functions of the present disclosure are possible and should be understood. First, surgical procedures can be performed open, mini-open, or arthroscopically. Second, all inside graft techniques (e.g., in situ techniques), multiple bundle constructs, and graft fixations can be performed in any manner, such as with sutures directly into bone or adjacent tissue, tunnels, etc. Next, lower and upper connectors of the distraction device may be swapped (e.g., where the graft is attached to the upper or first connector, and bone is coupled to lower or second connector). Further, the term soft tissue structure encompasses grafts and ligaments. Also, while the description of the CAOS system of the present disclosure is described in a context with an ACL reconstruction procedure in a knee joint, the system and methods of the present disclosure can also be applied to other tissue structures, other repair/reconstruction procedures, and other joints and/or anatomical areas and procedures.

Still further, the systems and methods of the present disclosure could also be used to diagnose a problematic joint or instability, by quantifying a degree of joint laxity and/or soft tissue tension or elongation, to determine if a joint is unstable, and to determine which anatomical structures may be deficient and in need of repair or reconstruction. The distraction device may be used without a 3D tracking system, in a stand-alone mode, to measure and control graft tension and displacement/height. In certain aspects, multiple distraction devices may be used and controlled by the system of the present disclosure simultaneously. The bodies of the distraction devices may be connected to one another to form a single unit, or the bodies may be separated so they can be positioned relative to one another. Use of multiple distraction devices may be advantageous in multi-bundle ligament reconstructions, where tensions in multiple bundles or multiple grafts may need to be controlled separately, or at different orientations and positions from one another. In other aspects, communication between the distraction device, sensors, motors, displacement mechanisms, and controllers, and other components of the CAOS system may be wired or wireless (for example using Wi-Fi or Bluetooth protocols).

In accordance with another aspect of the present disclosure, a distraction device (such as the various distraction device embodiments discussed above) is configured to operate in a “stand-alone” mode (e.g., without being connected to an optical 3D tracking system or navigation system), and can be used with manual surgical instrumentation, such as manual cutting blocks. For example, a guide structure (e.g., guide structure 4102) can be coupled to the distraction device and configured to guide placement of a surgical instrument e.g., relative to a joint. In exemplary aspects, the distraction device can be used in a knee arthroplasty surgery and other orthopedic surgical intervention procedures (e.g., total or partial arthroplasty, soft tissue repairs, such as ligament reconstructions, tendon, labrum, muscle, capsule repairs, etc.), and in other procedures for joints and soft tissue structures, such as the spine, knee, hip, shoulder, elbow, ankle, pelvis, etc.

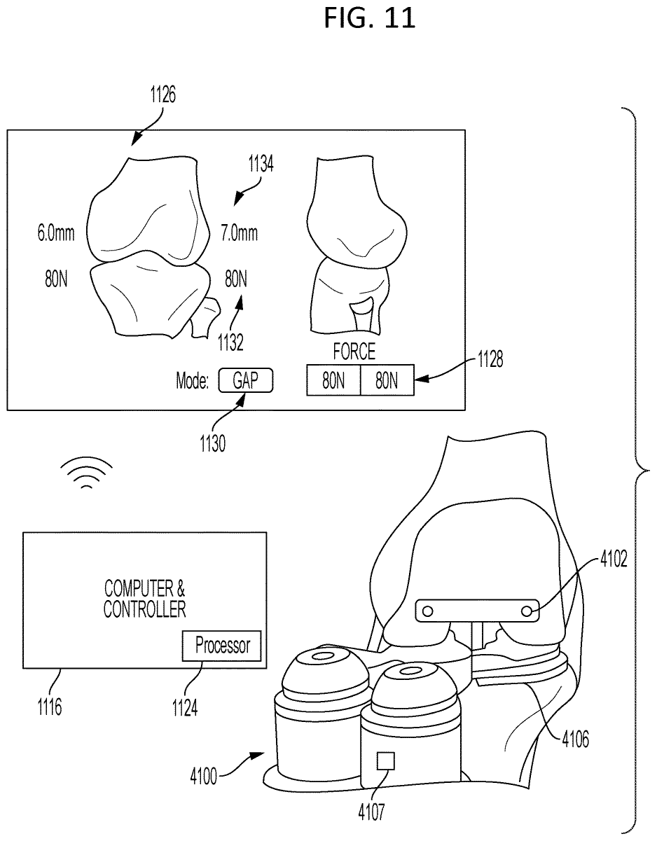

FIG. 11 illustrates a CAOS system including a computer and controller 1116, including a processer 1124, a tablet or a laptop with a touch screen, and a user interface (UI) 1126. The distraction device 4100 can be equipped with a guide structure 4102 (e.g., a drill guide or pin guides, and the like) that attach to a lower paddle/connector 4106, to secure cutting blocks to a bone of a joint e.g., a femur or tibia, such that resections can achieve a balanced joint. The UI includes interface buttons 1128 for setting or adjusting applied forces medially and laterally, and for switching a mode of the device from gap mode to height mode 1130, and vice versa. Symmetric or asymmetric tensions can be targeted (e.g., lower force laterally in flexion). Post resection, gap balance and joint forces can be checked and adjusted with trials. Thus, the user interface is configured to enable independent adjustment of medial and lateral forces on the joint by the distraction device as well as to selectively operate the distraction device in a gap-control mode or in a displacement-control or height-control mode.

A gyroscope 4107 can be integrated into the distraction device to measure angle relative to gravity, which can be used to estimate a flexion angle of the joint and provide force and gap data along with the flexion angle through the flexion range. The flexion angle of the joint can be estimated from an angle provided by the gyroscope, for example, by assuming a patient is lying supine, with their hip and ankle resting at a same vertical level; by assuming an average tibial and femoral length; and assuming that the distraction device rests on a largely perpendicular tibial resection (or on one of known slope, which can be factored in); or by assuming that the body of the distraction device is approximately parallel to the tibia. The UI can present measured gap data 1134 and measured force data 1132 for both the medial and lateral sides of the joint (or, just one side when a single distraction device is used).

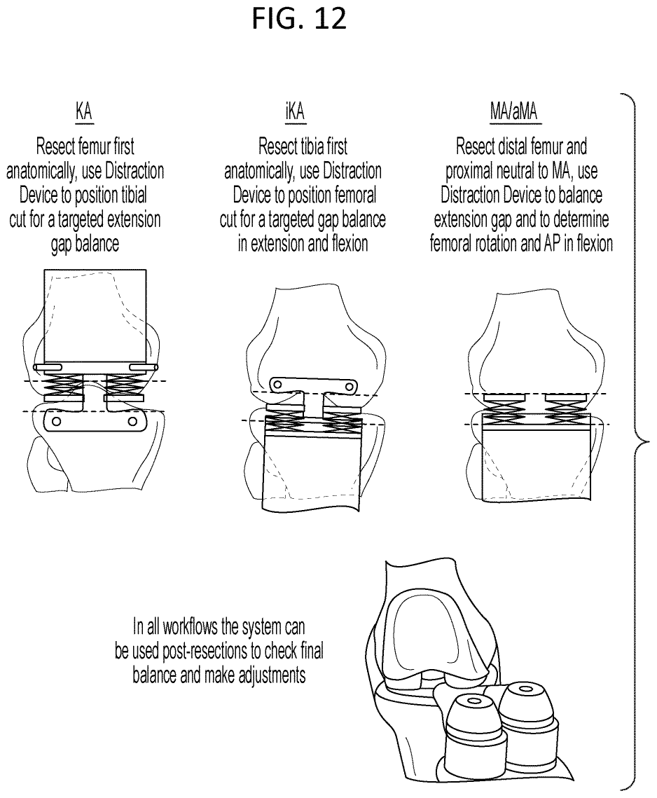

The CAOS system of the present disclosure could be designed to work with multiple workflows, including kinematic alignment (KA), inverse KA (iKA), mechanical alignment (MA), adjusted MA (aMA), gap balancing (GB), as well as partial arthroplasty applications (such as unicompartmental knee arthroplasty). FIG. 12 illustrates a summary of the KA, iKA and MA/aMA workflows.

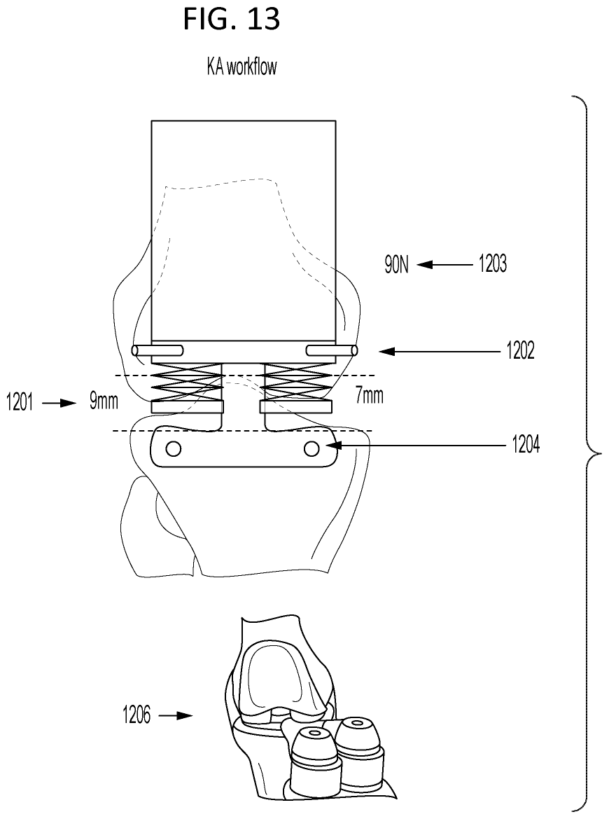

FIG. 13 illustrates one, non-limiting, embodiment of a kinematic alignment (KA) workflow for a knee joint, which can include some or all of the following, in non-restrictive order, summarized as follows:
a) Using a cutting block, resecting equal distal and posterior femoral resections after accounting for cartilage/bone wear;
b) Inserting the distraction device between the femur and tibia, using a slender upper and lower paddle of the distraction device, or by pinning the upper paddle of the distraction device to the femur;
c) Applying forces to a proximal tibia via the distraction device while the knee is in extension to distract the joint and to a set tension between the tibia and femur to a target force; the applied forces can be set to equal or unequal between the medial and lateral sides of the knee joint (e.g., higher forces can be applied to a medical side of the joint i.e., forces applied to the medical side of the joint are greater than forces applied to the lateral side of the joint);
d) Preparing guide holes within the tibia for receiving tibial guide pins once the target force is reached (as indicated on display);
e) Assembling a tibial cutting block onto the tibial guide pins, and manually resecting the tibia using the cutting block, the cutting block includes adjustment mechanism for dialing in tibial slope to match native slope, while maintaining consistent cut depth, or alternatively a knee extension angle can be adjusted to match native slope before tibial guide pins are pinned to the tibia;
f) Reinserting the distraction device after all bone cuts are made to perform a final gap and/or force balance in extension check, where the check can be completed as the joint is moved through a range of motion; and
g) Making adjustments to the bone resections if required based on characteristic parameter data obtained by the distraction device.

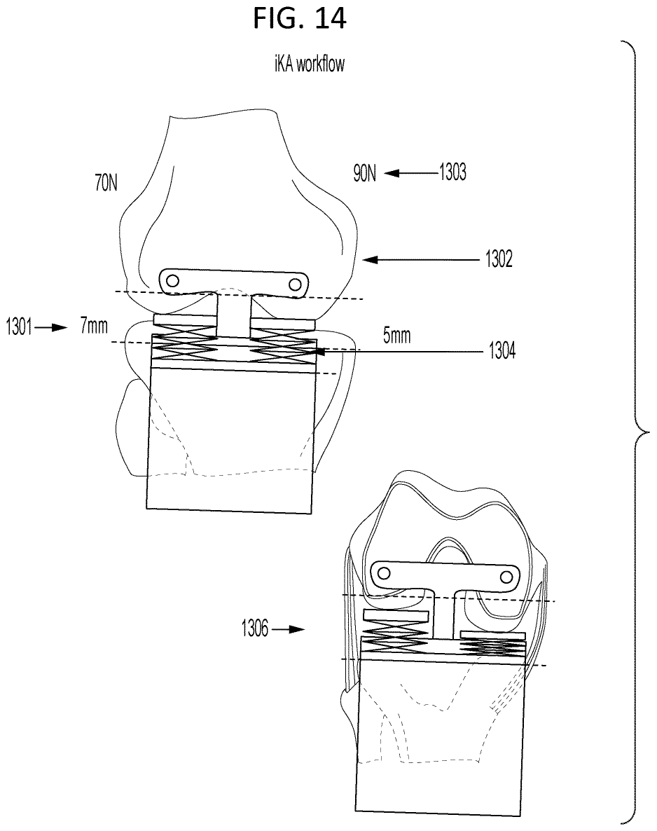

FIG. 14 illustrates one, non-limiting, embodiment of an inverse kinematic alignment (iKA) workflow for a knee joint, which can include some or all of the following, in non-restrictive order, summarized as follows:
a) Anatomically resecting the proximal tibia with equal medial and lateral resection depths after accounting for cartilage/bone wear using a conventional cutting block;
b) Inserting the distraction device between the tibia and femur of a knee joint; the distraction device includes a tibial baseplate having a drill guide attached and two upper paddles/connectors;
c) Applying forces to distal femur via the distraction device to distract the knee joint and to a set tension between the femur and tibia to a target force; applied forces can be set to equal or unequal between the medial and lateral sides of the knee joint (e.g., higher forces can be applied to a medical side of the joint i.e., forces applied to the medical side of the joint are greater than forces applied to the lateral side of the joint);
d) Preparing guide holes for receiving femoral guide pins after the target force is reached (as indicated on display);
e) Assembling a distal femoral cut-block onto the femoral guide pins and resecting the femur;
f) With the knee positioned at 90° flexion, applying distraction forces to the joint after the distraction device is reinserted, and determining femoral rotation and posterior resection levels with the distraction device distracting the femur from the tibia and with the ligaments stretched to a desired tension or predetermined characteristic parameter satisfies a predetermined target condition;
g) Drilling lug holes for a cutting block (e.g., a 4-in-1 cutting block) through the drill guide (which is sitting flush against the distal cut) to conform to the desired posterior resection level; the drill guide can be adapted depending on how the pegs/lug-holes are positioned amongst the various sizes in the implant system; the distraction device can also be integrated with an implant sizer to perform implant sizing, rotation and AP positioning simultaneously; and
h) Rendering AP cuts on the femur, inserting femoral trial components, and reinserting the distraction device to check final gap and/or force balance in extension and throughout range of motion of the joint; further resecting the femur for adjustments to bone resections, if required, based on characteristic parameter data obtained by the distraction device.

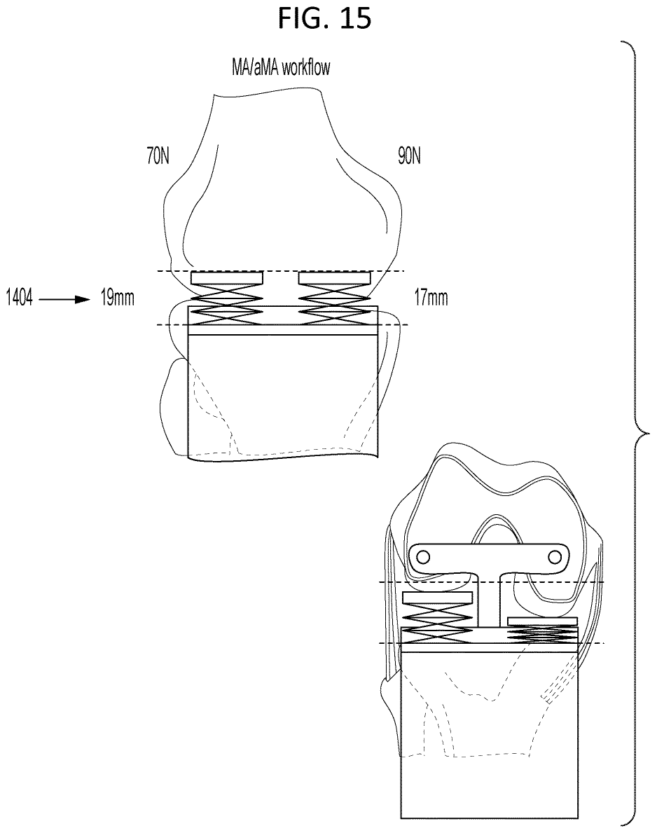

FIG. 15 illustrates one, non-limiting, embodiment of a mechanical alignment (MA)/ adjusted mechanical alignment (aMA) workflow for a knee joint, which can include some or all of the following, in non-restrictive order, summarized as follows:
a) Resecting the distal femur and proximal tibial in neutral or substantially neutral alignment, based on overall deformity and correctability of deformity (e.g., as in adjusted MA, aMA);
b) With knee in extension, inserting the distraction device between the femoral and tibial resections using existing or adapted upper and lower paddles/connectors (e.g., a spacer shim may be used to achieve heights beyond 20 mm);
c) Applying distraction device forces to a set, initial, or predefined tension between the femur and tibial to a target force; forces can be set to equal or unequal between the medial and lateral sides of the knee joint (e.g., higher forces can be applied to a medical side of the joint i.e., forces applied to the medical side of the joint are greater than forces applied to the lateral side of the joint);
d) Reporting spacing on either side of the joint via the distraction device, and performing releases on ligaments of the joint to balance the extension gap, if desired;
e) With the knee in flexion, reinserting the distraction device between the bones of the knee joint; rendering AP cuts on the bones of the joint to achieve a balanced flexion gap, as in the iKA technique (or rotation can be set using a measured resection technique, e.g., 3° external rotation);
f) Reinserting the distraction device after all bone cuts are made, with femoral trial in place, check final gap and/or force balance in extension and throughout range of motion; and
g) Making adjustments to tibial resection, if required, based on characteristic parameter data obtained by the distraction device.

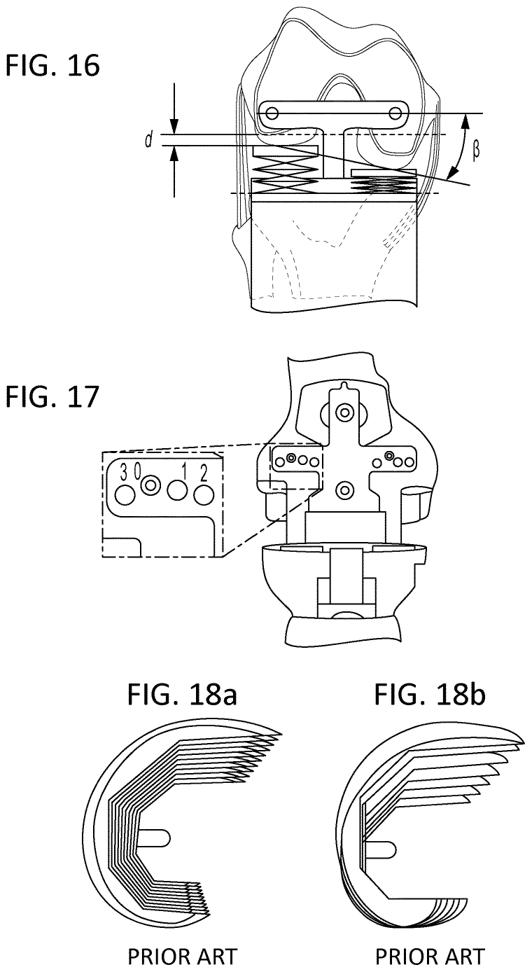

As shown in FIG. 16, in each workflow above, the distraction device can provide medial and lateral cutting depths d, when positioning resections because the distance between the upper paddles/connectors, and the drill guide holes are known. Angle β, between the cutting guide and the Posterior Condylar Axis (PCA), Lateral Distal Femoral Angle (LDFA), and Medial Proximal Tibial Axis (MPTA), can also be approximated and reported through use of geometric assumptions.

As shown in FIG. 17, multiple holes can be provided in the drill guide to provide variation in desired positioning, as in manual instruments, or to accommodate implants with different varying position of 4-in-1 peg holes.

FIGS. 18a and 18b illustrate a femoral implant with multiple sizes, where FIG. 8a shows an implant that grows with varying relationship between the pegs and posterior resections, and FIG. 18b shows an implant that grows with a consistent relationship between the pegs and posterior resections. The height of the paddles/connectors of the distraction device can be controlled to vary an implant size, while maintaining a target flexion gap balance and resection level. The CAOS system of the present disclosure can store the position at a desired resection depth and angle, and then move paddles up or down symmetrically to drill 4-in-1 holes or implant pegs for an implant size, up or down. This can be used in combination with multiple drill guide holes. In other words, the controller of the subject CAOS system is configured to determine an implant size based on a measured displacement e.g., a measured displacement via the distraction device. Therefore, the system of the present disclosure could cover an entire implant size range with a single drill guide template.

### Features of the Invention

1. An orthopedic system for conditioning a soft tissue structure, comprising:
a distraction device including:
   a first connector configured to engage a first anatomical structure associated with a soft tissue structure,
   a second connector configured to engage a second anatomical structure associated with the soft issue structure, and
   a displacement mechanism configured to apply a force to one of the first and second connectors for applying a tension to the soft tissue structure;
at least one sensor configured to generate parameter measurement data associated with the soft issue structure and/or the distraction device upon the displacement mechanism applying the force to one of the first and second connectors, the parameter measurement data comprising at least one of a force, and a displacement of the soft tissue structure; and
a controller operatively associated with the distraction device and the at least one sensor, wherein the controller is configured to:
   receive parameter measurement data from the at least one sensor,
   determine a characteristic parameter of the soft tissue structure based on the parameter measurement data, and
   control the displacement mechanism in response to the determined characteristic parameter until the determined characteristic parameter satisfies a predetermined target condition.

2. The system of feature 1, wherein the anatomical structure is hard tissue or soft tissue, and wherein the soft tissue structure is a graft, a ligament, a tendon, a muscle, cartilage, capsular tissue,meniscus, fascia, or connective tissue.

3. The system of feature 1, wherein the parameter measurement data is a force or a displacement, and wherein the characteristic parameter is a tension force, a percentage elongation, a change in elongation, a length, an overall length, or a thickness.

4. The system of feature 1, wherein the distraction device is configured to engage the first and second anatomical structures in situ or ex situ.

5. The system of feature 1, wherein the controller is configured to control the displacement mechanism by updating the force applied to one of the first and second connectors.

6. The system of feature 1, wherein the controller is configured to control the displacement mechanism by applying a cyclical force to one of the first and second connectors.

7. The system of feature 6, wherein the cyclical force is applied over a predefined time period.

8. The system of feature 1, wherein the target condition comprises the characteristic parameter of the soft tissue structure having a delta measurement less than a predefined amount after successive updates to the control of the displacement mechanism.

9. The system of feature 8, wherein the predefined amount is 1-40% of the characteristic parameter.

10. The system of feature 1, wherein the controller is configured to receive parameter measurement data from the at least one sensor as the first and second anatomical structures are moved between first and second positions.

11. The system of feature 10, further comprising a 3D tracking system configured to determine a position of the first and second anatomical structures.

12. The system of feature 1, wherein the controller determines a surgical plan based on the characteristic parameter at the target condition.

13. A system for tensioning a soft tissue structure of a joint, comprising:
a distraction device including:
   a first connector configured to engage a first anatomical structure associated with a soft tissue structure,
   a second connector configured to engage a second anatomical structure associated the soft tissue structure, and
   a displacement mechanism operable to displace the first connector relative to the second connector;
at least one sensor configured to measure a parameter associated with the soft tissue structure and/or the distraction device, the parameter comprising at least one of a force, a tension, an elongation and a displacement of the soft tissue structure; and
a controller operatively in communication with the distraction device and the at least one sensor, the controller configured to:
   control the displacement mechanism to displace the first connector relative to the second connector upon engagement with the first and/or the second anatomical structures associated with the soft tissue structure, and apply a predetermined force to displace the first connector relative to the second connector,
   measure a tension of the soft tissue structure upon application of the predetermined force to displace the first connector relative to the second connector,
   determine a displacement height of the first and second connectors corresponding to the measured tension of the soft tissue structure reaching a predetermined tension, and
   measure a tension of the soft tissue structure as the soft tissue structure moves between first and second positions while the controller holds the displacement height of the first and second connectors at the determined height.

14. The system of feature 13, further comprising a navigation system to track a position of the first anatomical structure and the second anatomical structure.

15. The system of feature 13, further comprising a display for displaying at least one of force data, tension data, elongation data and displacement data of the soft tissue structure relative to a position of the first and/or second anatomical structure.

16. A system for measuring soft tissue structures of a joint, the system comprising:
a distraction device including:
   a first connector configured to engage a first bone of the joint or a soft tissue structure associated with the first bone of the joint,
   a second connector configured to engage a second bone of the joint or a soft tissue structure associated with the second bone of the joint, and
   a displacement mechanism operable to displace the first connector relative to the second connector;
at least one sensor configured to measure a parameter associated with the soft tissue structure and/or the distraction device, the parameter comprising at least one of a force and a displacement of the soft tissue structure; and
a controller operatively in communication with the distraction device and the at least one sensor, the controller configured to:
   control the displacement mechanism and apply a predetermined force to displace the first connector relative to the second connector of the distraction device upon engagement of the first and second connectors with the first and second bones or the soft tissue structure associated with the first bone or the second bone,
   determine a displacement between the first and second connectors and a degree of elongation of the soft tissue structure upon application of the predetermined force to the displacement mechanism, and
   display the degree of elongation of the soft tissue structure on a display.

17. The system of feature 16, further comprising a navigation system configured to track a position of the first bone, the second bone, and/or soft tissue structure.

18. The system of feature 16, wherein the at least one sensor is configured to measure at least one of force data, tension data, elongation data and/or displacement data of the soft tissue structure.

19. The system of feature 16, wherein the controller is further configured to display an elongation profile of the soft tissue structure as a function of joint position over a range of motion.

20. The system of feature 16, wherein the controller is further configured to quantify a laxity assessment of the joint while applying the predetermined force.

21. A system for performing an orthopedic surgical procedure, comprising:
a distraction device configured to apply a controlled force or displacement between first and second bones of a joint;
at least one sensor configured to measure at least one of force and displacement;
a user interface configured to:
   display the measured force and/or displacement, and
   receive user input to control the applied controlled force and/or displacement; and
a guide structure coupled to the distraction device and configured to guide placement of a surgical instrument relative to the joint; and
a controller operatively in communication with the distraction device, the at least one sensor and the user interface and configured to position the surgical instrument based on the measured force and/or the measured displacement applied by the distraction device.

22. The system of feature 21, wherein the user interface is configured to selectively operate the distraction device in a force-control mode or in a displacement-control mode.

23. The system of feature 21, wherein the user interface is configured to enable independent adjustment of medial and lateral forces on the joint by the distraction device.

24. The system of feature 21, wherein the controller is configured to determine an implant size based on the measured displacement.

It will be appreciated by those skilled in the art that changes could be made to the various aspects described above without departing from the broad inventive concept thereof. It is to be understood, therefore, that the subject application is not limited to the particular aspects disclosed, but it is intended to cover modifications within the spirit and scope of the subject application as defined by the appended claims.

I/We claim:

1. An orthopedic system for conditioning a soft tissue structure, comprising:
a distraction device including:
a first connector configured to engage a first anatomical structure associated with a soft tissue structure,
a second connector configured to engage a second anatomical structure associated with the soft issue structure, and
a displacement mechanism configured to apply a force to one of the first and second connectors for applying a tension to the soft tissue structure;
at least one sensor configured to generate parameter measurement data associated with the soft issue structure and/or the distraction device upon the displacement mechanism applying the force to one of the first and second connectors, the parameter measurement data comprising at least one of a force, and a displacement of the soft tissue structure; and
a controller operatively associated with the distraction device and the at least one sensor, wherein the controller is configured to:
receive parameter measurement data from the at least one sensor,
determine a characteristic parameter of the soft tissue structure based on the parameter measurement data, and
control the displacement mechanism in response to the determined characteristic parameter until the determined characteristic parameter satisfies a predetermined target condition.

2. The system of claim 1, wherein the anatomical structure is hard tissue or soft tissue, and wherein the soft tissue structure is a graft, a ligament, a tendon, a muscle, cartilage, capsular tissue,meniscus, fascia, or connective tissue.

3. The system of claim 1, wherein the parameter measurement data is a force or a displacement, and wherein the characteristic parameter is a tension force, a percentage elongation, a change in elongation, a length, an overall length, or a thickness.

4. The system of claim 1, wherein the distraction device is configured to engage the first and second anatomical structures in situ or ex situ.

5. The system of claim 1, wherein the controller is configured to control the displacement mechanism by updating the force applied to one of the first and second connectors.

6. The system of claim 1, wherein the controller is configured to control the displacement mechanism by applying a cyclical force to one of the first and second connectors.

7. The system of claim 6, wherein the cyclical force is applied over a predefined time period.

8. The system of claim 1, wherein the target condition comprises the characteristic parameter of the soft tissue structure having a delta measurement less than a predefined amount after successive updates to the control of the displacement mechanism.

9. The system of claim 8, wherein the predefined amount is 1-40% of the characteristic parameter.

10. The system of claim 1, wherein the controller is configured to receive parameter measurement data from the at least one sensor as the first and second anatomical structures are moved between first and second positions.

11. The system of claim 10, further comprising a 3D tracking system configured to determine a position of the first and second anatomical structures.

12. The system of claim 1, wherein the controller determines a surgical plan based on the characteristic parameter at the target condition.

13. A system for tensioning a soft tissue structure of a joint, comprising:
a distraction device including:
a first connector configured to engage a first anatomical structure associated with a soft tissue structure,
a second connector configured to engage a second anatomical structure associated the soft tissue structure, and
a displacement mechanism operable to displace the first connector relative to the second connector;
at least one sensor configured to measure a parameter associated with the soft tissue structure and/or the distraction device, the parameter comprising at least one of a force, a tension, an elongation and a displacement of the soft tissue structure; and
a controller operatively in communication with the distraction device and the at least one sensor, the controller configured to:
control the displacement mechanism to displace the first connector relative to the second connector upon engagement with the first and/or the second anatomical structures associated with the soft tissue structure, and apply a predetermined force to displace the first connector relative to the second connector,
measure a tension of the soft tissue structure upon application of the predetermined force to displace the first connector relative to the second connector,
determine a displacement height of the first and second connectors corresponding to the measured tension of the soft tissue structure reaching a predetermined tension, and
measure a tension of the soft tissue structure as the soft tissue structure moves between first and second positions while the controller holds the displacement height of the first and second connectors at the determined height.

14. The system of claim 13, further comprising a navigation system to track a position of the first anatomical structure and the second anatomical structure.

15. The system of claim 13, further comprising a display for displaying at least one of force data, tension data, elongation data and displacement data of the soft tissue structure relative to a position of the first and/or second anatomical structure.

16. A system for measuring soft tissue structures of a joint, the system comprising:
a distraction device including:
a first connector configured to engage a first bone of the joint or a soft tissue structure associated with the first bone of the joint,
a second connector configured to engage a second bone of the joint or a soft tissue structure associated with the second bone of the joint, and
a displacement mechanism operable to displace the first connector relative to the second connector;
at least one sensor configured to measure a parameter associated with the soft tissue structure and/or the distraction device, the parameter comprising at least one of a force and a displacement of the soft tissue structure; and
a controller operatively in communication with the distraction device and the at least one sensor, the controller configured to:
control the displacement mechanism and apply a predetermined force to displace the first connector relative to the second connector of the distraction device upon engagement of the first and second connectors with the first and second bones or the soft tissue structure associated with the first bone or the second bone,
determine a displacement between the first and second connectors and a degree of elongation of the soft tissue structure upon application of the predetermined force to the displacement mechanism, and
display the degree of elongation of the soft tissue structure on a display.

17. The system of claim 16, further comprising a navigation system configured to track a position of the first bone, the second bone, and/or soft tissue structure.

18. The system of claim 16, wherein the at least one sensor is configured to measure at least one of force data, tension data, elongation data and/or displacement data of the soft tissue structure.

19. The system of claim 16, wherein the controller is further configured to display an elongation profile of the soft tissue structure as a function of joint position over a range of motion.

20. The system of claim 16, wherein the controller is further configured to quantify a laxity assessment of the joint while applying the predetermined force.

21. A system for performing an orthopedic surgical procedure, comprising:
a distraction device configured to apply a controlled force or displacement between first and second bones of a joint;
at least one sensor configured to measure at least one of force and displacement;
a user interface configured to:
display the measured force and/or displacement, and
receive user input to control the applied controlled force and/or displacement; and
a guide structure coupled to the distraction device and configured to guide placement of a surgical instrument relative to the joint; and
a controller operatively in communication with the distraction device, the at least one sensor and the user interface and configured to position the surgical instrument based on the measured force and/or the measured displacement applied by the distraction device.

22. The system of claim 21, wherein the user interface is configured to selectively operate the distraction device in a force-control mode or in a displacement-control mode.

23. The system of claim 21, wherein the user interface is configured to enable independent adjustment of medial and lateral forces on the joint by the distraction device.

24. The system of claim 21, wherein the controller is configured to determine an implant size based on the measured displacement.
